(19) United States
(12) Patent Application Publication
(10) Pub. No.: US 2006/0223877 A1
(43) Pub. Date: Oct. 5, 2006

(54) Methods of treatment utilizing certain melatonin derivatives

(76) Inventor: Frank P. Zemlan, Cincinnati, OH (US)

Correspondence Address: FROST BROWN TODD, LLC, 2200 PNC CENTER, 201 E. FIFTH STREET, CINCINNATI, OH, 45202, US

(21) Appl. No.: 11/384,772

(22) Filed: Mar. 20, 2006

Related U.S. Application Data
(60) Provisional application No. 60/666,954, filed on Mar. 31, 2005.

(51) Int. Cl. A61K 31/405

(52) U.S. Cl. 514/419

(57) ABSTRACT

A method for treating anxiety disorders, affective disorders, intracranial injury, spinal cord injury, neurodegenerative diseases, sclerosis, migraine, fibromyalgia and cerebrovascular disease, using melatonin derivatives is disclosed. A specific melatonin derivative for use in the disclosed process is 13-methyl-6-chloromelatonin (otherwise referred to as (R)-N-[2-(6-chloro-5-methoxy-1H-indol-3-yl)propyl]acetamide).

### CROSS-REFERENCE TO RELATED APPLICATION

This application is related to and claims priority from U.S. Provisional Patent Application 60/666,954, Zemlan, filed Mar. 31, 2005, incorporated herein by reference.

### TECHNICAL FIELD

The present application relates to the use of melatonin derivatives to treat several medical conditions, as defined herein.

### SUMMARY OF THE INVENTION

The present invention relates to a method of treating a medical condition selected from anxiety disorders, affective disorders, obesity, intracranial injury, spinal cord injury, dementia of the Alzheimer's type, Parkinson's disease, sclerosis, migraines, fibromyalgia, and cerebrovascular disease, by administering to a patient in need of such treatment a safe and effective amount of melatonin derivative selected from: 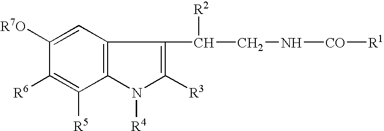
wherein

R¹ is hydrogen, C₁-C₄ alkyl or C₁-C₄ alkoxy;

R² is hydrogen or C₁-C₄ alkyl;

R³ is hydrogen, C₁-C₄ alkyl, phenyl or substituted phenyl;

R⁴ is hydrogen, haloacetyl, C₁-C₅ alkanoyl, benzoyl or benzoyl substituted with halo or methyl;

R⁵ and R⁶ are each individually hydrogen or halo; and

R⁷ is hydrogen or C₁-C₄ alkyl;

provided that when R³, R⁴ and R⁵ are each hydrogen, then R² must be C₁-C₄ alkyl.

### BRIEF DESCRIPTION OF THE DRAWINGS

FIG. 1 reports the results of a test demonstrating use of the present invention in providing an antidepressant effect (in terms of rearing behavior).

FIG. 2 reports the results of a test demonstrating use of the present invention in providing an antidepressant effect (in terms of immobility behavior).

FIG. 3 reports the results of a test demonstrating use of the present invention in treating traumatic brain injury.

FIG. 4 reports the results of a test demonstrating use of the present invention in treating neurodegenerative diseases.

FIG. 5 reports the results of a test demonstrating use of the present invention in treating neurodegenerative diseases.

FIG. 6 reports the results of a test demonstrating use of the present invention in treating cerebrovascular disease.

### DETAILED DESCRIPTION OF THE INVENTION

The melatonin derivatives used in the present invention are known. One group, described in U.S. Pat. No. 5,654,325, Flaugh, issued Aug. 5, 1997, incorporated by reference herein, has the following formula: 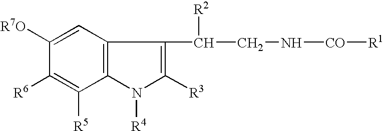
wherein

R¹ is hydrogen, C₁-C₄ alkyl or C₁-C₄ alkoxy;

R² is hydrogen or C₁-C₄ alkyl;

R³ is hydrogen, C₁-C₄ alkyl, phenyl or substituted phenyl;

R⁴ is hydrogen, haloacetyl, C₁-C₅ alkanoyl, benzoyl or benzoyl substituted with halo or methyl;

R⁵ and R⁶ are each individually hydrogen or halo; and

R⁷ is hydrogen or C₁-C₄ alkyl;

provided that when R³, R⁴ and R⁵ are each hydrogen, then R² must be C₁-C₄ alkyl.

In one embodiment, compounds for use in the methods of treatment claimed herein include compounds wherein R¹ is C₁-C₄ alkyl (especially methyl), R³ is hydrogen or C₁-C₄ alkyl (especially methyl), and R⁴ is hydrogen. Of such compounds, another embodiment includes those compounds wherein R² and R⁷ are each independently C₁-C₄ alkyl (preferably methyl). Examples of such compounds include N-[2-methyl-2-(5-methoxy-6-fluoroindol-3-yl)ethyl]acetamide, N-[2-ethyl-2-(5-methoxy-6-chloroindol-3-yl)ethyl]acetamide, N-[2-methyl-2-(5-methoxy-6,7-dichloroindol-3-yl)ethyl]acetamide and N-[2-methyl-2-(5-methoxy-6-chloroindol-3-yl)ethyl]acetamide, and mixtures of the compounds.

A specific compound for use in the present invention is β-methyl-6-chloromelatonin (otherwise referred to as (R)-N-[2-(6-chloro-5-methoxy-1H-indol-3-yl)propyl]acetamide).

These compounds are well known and can be made by methods disclosed in the art. Representative publications which teach the preparation of these compounds include U.S. Pat. No. 4,087,444, Flaugh et al., issued May 2, 1978; U.S. Pat. No. 4,614,807, Flaugh, issued Sep. 30, 1986; and U.S. Pat. No. 4,997,845, Flaugh, issued Mar. 5, 1991; all of which are incorporated herein by reference.

The melatonin derivatives described herein can be used to treat the following conditions (all of which are described in the Diagnostic and Statistical Manual, Fourth Edition (DSM-IV), published by the American Psychiatric Association, Washington, D.C., 2000, or in The ICD-10 Classification of Mental and Behavioral Disorders: Clinical Descriptions and Diagnostic Guidelines, published by the World Health Organization (WHO), Geneva, 1992):
anxiety disorders—including panic attack, obsessive-compulsive disorder, post-traumatic stress disorder, generalized anxiety disorder;
affective disorders—including bipolar disorders, depression, major depressive disorder, dysthymic disorder;
intracranial injury—including traumatic brain injury, closed head injury, open head injury;
spinal cord injury;
obesity—including morbid obesity;
neurodegenerative diseases—including dementia of the Alzheimer's type (including Alzheimer's disease) and Parkinson's disease;
sclerosis—including amyotrophic lateral and multiple sclerosis;
migraine—including classical migraine, common migraine, cluster headaches, neuralgia;
fibromyalgia; and
cerebrovascular disease—including subarachnoid hemorrhage, intracerebral hemorrhage, cerebral infarction, stroke, and cerebral aneurysm.

As described herein, the present invention provides a method of treating specified mental and central nervous system disorders utilizing specifically-defined melatonin analogs. The claimed melatonin analogs demonstrate significant affinity for melatonin receptors. For example, one compound of the present invention, β-methyl-6-chloromelatonin, demonstrates high affinity binding to melatonin receptors (Mulchahey, et al., 2004). The present method of treatment is believed to be more effective in terms of efficacy, duration of action and side effects than previous methods known for treating said disorders. Additionally, the melatonin analogs of the present invention are believed to be without toxicity at the preferred treatment dosages (20 mg to 100 mg of the active ingredient per day) and, as such, represent a significant improvement in the treatment of said disorders. For example, treatment of humans with 20 mg to 100 mg per day of a compound of the present invention, β-methyl-6-chloromelatonin, resulted in no significant side effects compared to placebo treatment (Zemlan et al., 2005).

References

Mulchahey, J., et al. (2004). A single blind, placebo controlled, across groups dose escalation study of the safety, tolerability, pharmacokinetics and pharmacodynamics of the melatonin analog beta-methyl-6-chloromelatonin. Life sciences, 75(15), 1843-1856.

Zemlan, F., et al. (2005). The efficacy and safety of the melatonin agonist beta-methyl-6-chloromelatonin in primary insomnia: a randomized, placebo-controlled, crossover clinical trial. The Journal of clinical psychiatry, 66(3), 384-390.

### Obesity

Melatonin is an effective treatment for obesity (Barrenetxe et al., 2004). In a preclinical model of human diet-induced obesity, daily melatonin administration significantly decreased body weight in subjects fed a high-fat diet (Pruet-Marcassus et al., 2003). This significant decrease in body weight was observed as soon as 5 days after the initiation of daily melatonin treatment and continued through the entire time-course of melatonin treatment. In addition to melatonin's efficacy in treating diet-induced obesity, melatonin is also effective in treating middle-age obesity (Wolden-Hanson et al., 2000). For example, daily treatment with melatonin significantly decreased body weight in a preclinical model of middle-aged obesity (Rasmussen et al., 1999). Importantly, this melatonin-induced decrease in middle-age obesity was due to a significant decrease in fat content as opposed to lean body mass, further indicating the effectiveness of melatonin for the treatment of obesity. The melatonin derivatives described herein are effective for treating obesity.

References

Barrenetxe, J., et al. (2004). Physiological and metabolic functions of melatonin. Journal of physiology and biochemistry, 60(1), 61-72.

Prunet-Marcassus, B., et al. (2003). Melatonin reduces body weight gain in Sprague Dawley rats with diet-induced obesity. Endocrinology, 144(12), 5347-5352.

Rasmussen, D., et al. (1999). Daily melatonin administration at middle age suppresses male rat visceral fat, plasma leptin, and plasma insulin to youthful levels. Endocrinology, 140(2), 1009-1012.

Wolden-Hanson, T., et al. (2000). Daily melatonin administration to middle-aged male rats suppresses body weight, intra-abdominal adiposity, and plasma leptin and insulin independent of food intake and total body fat. Endocrinology, 141(2), 487-497.

### Migraines

Melatonin has been shown to be an effective treatment for migraines and other types of headaches (Gagnier, 2001; Peres, 2005). For example, patients with a diagnosis of migraine with or without aura were treated daily with melatonin (Peres et al., 2004). In this study, melatonin treatment resulted in a significant decrease in headache frequency, as well as a decrease in headache intensity, clearly indicating the efficacy of melatonin for the treatment of migraine. The melatonin derivatives described herein are similarly effective for the treatment of migraines.

References

Gagnier, J. J. (2001). The therapeutic potential of melatonin in migraines and other headache types. Alternative medicine review: a journal of clinical therapeutic, 6(4), 383-389.

Peres, M. F., et al., (2004). Melatonin, 3 mg, is effective for migraine prevention. Neurology, 63(4), 757.

Peres, M. F. P. (2005). Melatonin, the pineal gland and their implications for headache disorders. Cephalalgia : an international journal of headache, 25(6), 403-411.

### Fibromyalgia

Melatonin has been shown to be an effective treatment for fibromyalgia. In a recent study, 20 patients with a diagnosis of fibromyalgia were treated for 30 days with melatonin (Citera et al., 2000). Significant improvement in the core symptoms of fibromyalgia were observed including improvement in severity of pain and tender point count, as well as more positive patient-ratings and physician-ratings of clinical improvement. In a similar study, fibromyalgia patients were treated daily with melatonin (Acuna-Castroviejo et al., 2006). At the end of treatment, all patients in this study reported significant improvement including lack of pain and fatigue, two cardinal symptoms of fibromyalgia. The melatonin derivatives described herein are similarly effective for the treatment of fibromyalgia.

References

Acuna-Castroviejo, D., et al. (2006) Melatonin therapy in fibromyalgia. Journal of pineal research: 40(1):98-9.

Citera, G., et al. (2000). The effect of melatonin in patients with fibromyalgia: a pilot study. Clinical rheumatology, 19(1), 9-13.

### Affective Disorders and Anxiety Disorders

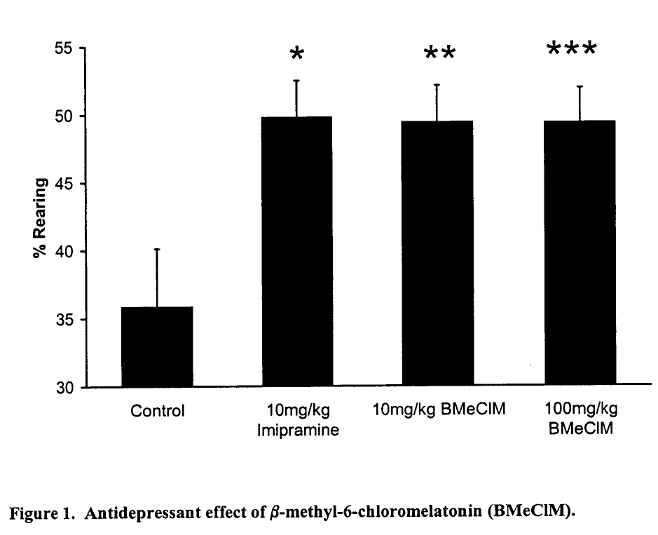

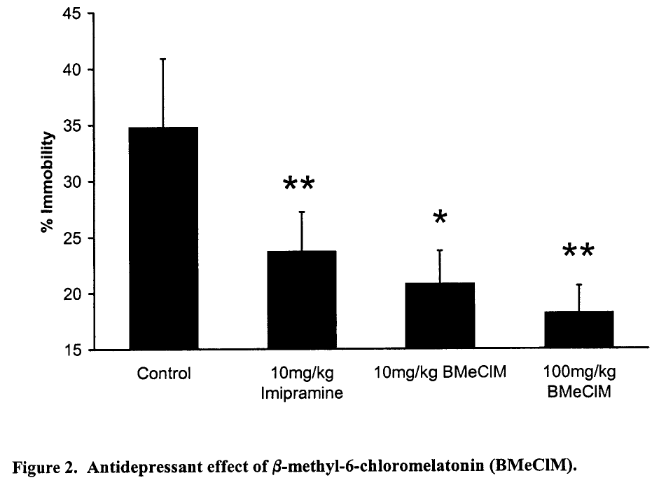

The compounds of the present invention are effective in treating affective disorders (depression, major depressive disorders, dysthymic disorders and bipolar disorders) and anxiety disorders (generalized anxiety disorder, panic attack, obsessive-compulsive disorder and post-traumatic stress disorder). The efficacy of the compounds of the present invention was demonstrated employing the open field test which is a well-accepted preclinical model of emotional disorders, including affective disorders and anxiety disorders (Ramos and Mormede, 1998). As shown in FIGS. 1 and 2, compounds such as imipramine, which is approved by the U.S. Food and Drug Administration (FDA) for the treatment of affective disorders in humans, significantly decrease rearing behavior and increase immobility behavior in the open field test (Physicians' Desk Reference, 2006). Therefore, compounds that decrease rearing behavior and increase immobility behavior in the open field test are considered to be effective treatments for affective disorders and anxiety disorders in this preclinical model (Ramos and Mormede, 1998). The efficacy of one compound of the present invention was demonstrated according to the following protocol.

Subjects were male Sprague-Dawley rats weighing 250-300 g housed in a temperature- and humidity-controlled vivarium on a 12:12 hr light:dark cycle with food and water available ad libitum. Behavioral testing occurred 2 hrs after lights out. Subjects were randomly assigned to the three treatments: β-methyl-6-chloro-melatonin, the FDA-approved antidepressant imipramine, or vehicle control. β-methyl-6-chloromelatonin was administered at two doses: 10 and 100 mg/kg i.p., imipramine at 10 mg/kg i.p., and vehicle i.p. at a comparable volume, all 1 hr before open field testing. All treatments were administered to an animal once (no repeat drug administration) with 8 animals per treatment.

The open filed testing procedure has been previously described (Herman et al., 2003). Briefly, the open field apparatus is a 36×36-inch white PLEXIGLAS® enclosure, divided into 36 squares of equal size. Animals are placed into the apparatus and allowed to explore the environment for 5 min. Sessions are videotaped and scored for behavior by an investigator blinded to the treatment condition. Behavior is scored for the two primary outcome measures: rearing and immobility, as well as grooming; and secondary measures of sedation: total mobility and quadrant crossing.

The effect of β-methyl-6-chloromelatonin (10 and 100 mg/kg, i.p. 1 hr prior to open field testing), imipramine (10 mg/kg, i.p. 1 hr prior to open field testing) and vehicle control on open field behavior was determined. Rearing and immobility are considered reliable measures of antidepressant activity (that is, drugs that are approved by the USFDA for the treatment of depression in humans increase rearing and decrease immobility) (FIGS. 1 and 2). In the present study, both the 10 and 100 mg/kg β-methyl-6-chloromelatonin doses significantly increased rearing behavior compared to vehicle controls (P=0.006 for both, FIG. 1). Similarly, the FDA-approved antidepressant imipramine significantly increased rearing behavior (P=0.005). Regarding immobility, both the 10 and 100 mg/kg β-methyl-6-chloro-melatonin significantly decreased immobility compared to controls (P=0.011 and 0.003, respectively). Similarly, the FDA-approved antidepressant imipramine significantly decreased immobility (P=0.043). See FIG. 2. Neither dose of β-methyl-6-chloromelatonin produced a sedative effect as there was no significant drug effect on total mobility or quadrant crossings (P values>0.10). These data indicate that β-methyl-6-chloromelatonin demonstrates significant and reliable antidepressant/antianxiety effects in this well-established preclinical model at doses that are not sedative.

References

Herman, J., et al. (2003). Norepinephrine-gamma-aminobutyric acid (GABA) interaction in limbic stress circuits: effects of reboxetine on GABAergic neurons. Biological psychiatry, 53(2), 166-174.

Physicians' Desk Reference. 60th ed., Thomson Healthcare, Inc.: Montvale (N.J.), 2006, p. 2491.

Ramos, A., & Mormede, P. (1998). Stress and emotionality: a multidimensional and genetic approach. Neuroscience and biobehavioral reviews, 22(1), 33-57.

Central Nervous System Injuries Including Intracranial Injury and Spinal Cord Injury

The compounds of the present invention are effective in treating injuries of the central nervous system, including intracranial injury also referred to as traumatic brain injury (TBI) and spinal cord injury (SCI). The efficacy of the compounds of the present invention was demonstrated employing a well-accepted preclinical model of TBI (Facchinetti et al., 1998; Chen et al., 2003). The efficacy of one compound of the present invention was demonstrated according to the following protocol.

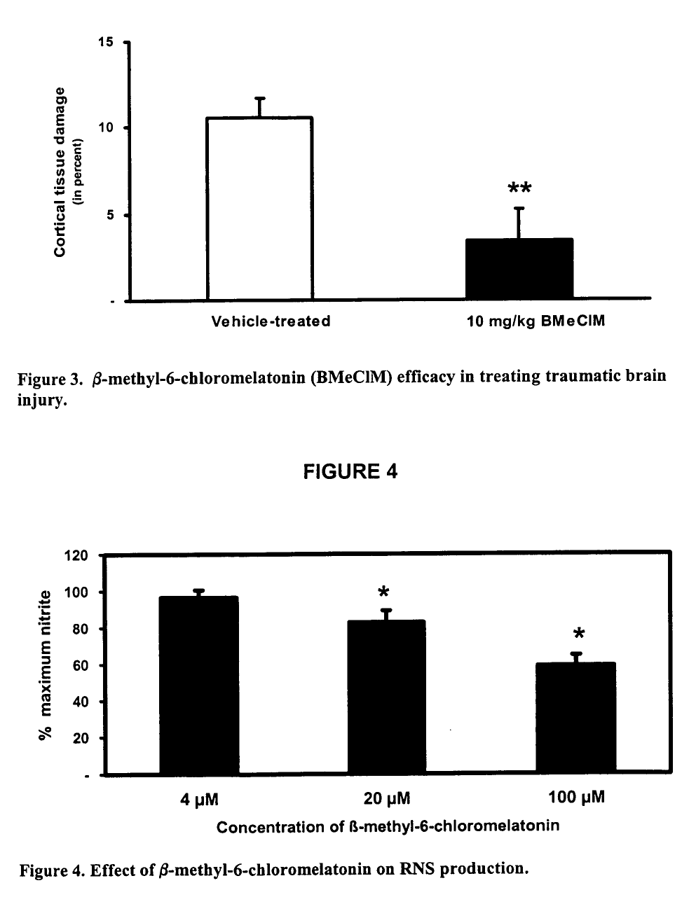

Subjects were male Sprague-Dawley rats weighing 250-300 g housed in a temperature- and humidity-controlled vivarium on a 12:12 hr light:dark cycle with food and water available ad libitum. Animals were subjected to TBI using controlled cortical impact model. (Sullivan et al. 2000a). Animals were anesthetized and their brain cortex exposed. Employing a pneumatically-controlled impactor rod with a 5 mm diameter beveled tip, one of the cortices was compressed at 3.5 m/sec to a predetermined depth of 1.5 mm, resulting in TBI. The other cortex was not injured and left intact. Following the experimental TBI protocol, the animals were randomly divided into two groups and were either treated with vehicle or 10 mg/kg of β-methyl-6-chloromelatonin intraperitoneally (two doses; first dose 15 minutes after TBI and the second 24 hours later). The animals were allowed to recover for 168 hours (seven days). On Day 7, quantitative morphometric image analysis was employed to assess cortical tissue damage. Quantitative morphometry is considered to be the “gold standard” for assessing neuroprotectant drug efficacy in TBI (Sullivan et al., 1999; Sullivan et al., 2000a; Sullivan et al., 2000b). Percent tissue damage was calculated based on the amount of intact tissue in the injured cortex normalized to intact tissue present in the uninjured cortex. β-methyl-6-chloromelatonin treatment resulted in a highly significant 68% reduction in damaged cortical tissue in TBI rats compared to vehicle-treated TBI rats (* P=0.01; FIG. 3). These data indicate that β-methyl-6-chloromelatonin blocks TBI-associated cortical tissue damage and is an effective treatment for TBI in this preclinical model.

References

Facchinetti, F., et al. (1998) Free radicals as mediators of neuronal injury. Cell Mol. Neurobiol., 18(6): 667-82.

Chen, S., et al. (2003) Time course of cellular pathology after controlled cortical impact injury. Exp. Neurol., 182(1): 87-102.

Sullivan, P. G., et al. (1999) Cyclosporin A attenuates acute mitochondrial dysfunction following traumatic brain injury. Exp. Neurol., 160(1): 226-34.

Sullivan, P. G., et al., (2000a) Continuous infusion of cyclosporin A postinjury significantly ameliorates cortical damage following traumatic brain injury in rodents. Exp. Neurol., 161(2): 631-7.

Sullivan, P. G., et al., (2000b) Dose-response curve and optimal dosing regimen of cyclosporin A after traumatic brain injury in rats. Neuroscience, 101(2): 289-95.

### Neurodegenerative Diseases

The compounds of the present invention are effective in treating neurodegenerative diseases, such as amyotrophic lateral sclerosis (ALS), Alzheimer's disease (AD), Huntington's disease (HD) and Parkinson's disease (PD). The efficacy of the compounds of the present invention was demonstrated employing EOC-20 microglial cells, a well-accepted microglial cell culture model of inflammation-induced neuronal injury such as ALS, AD, HD and PD (Hensley et al., 2003; West, 2004). The efficacy of a compound of the present invention was demonstrated according to the following protocol.

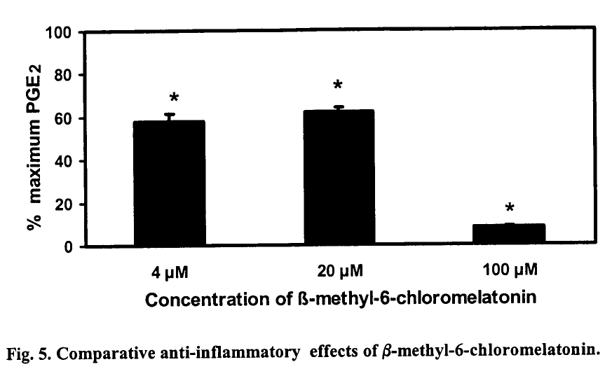

The experiments consisted of treating TNF-α stimulated EOC-20 cells with increasing concentrations of β-methyl-6-chloromelatonin followed by measuring neuroinflammation-associated markers. Neuroinflammation activates microglia to produce proinflammatory cytokines, reactive oxygen species (ROS) and reactive nitrogen species (RNS) in ALS, AD, HD and PD-associated pathophysiology (Deckel, 2001; Cacquevel, 2004; McGeer and McGeer, 2004; Sargsyanet al., 2005). Increase in prostaglandin (PGE₂), a potent inflammatory mediator, and nitrite, an indirect measure of RNS production, indicates inflammation-induced neurodegeneration in ALS, AD, HD and PD patients (Milstien et al., 1994; Tohgi et al., 1999; Deckel, 2001 ;Cacquevel, 2004). EOC-20 cells treated with 20 ng/ml TNF-α resulted in a significant increase in nitrite and PGE₂ levels (Hensley et al., 2003; West, 2004). β-methyl-6-chloromelatonin blocked nitrite production in 20 ng/ml TNF-α-stimulated EOC-20 cells in a dose-dependent manner (*P<0.01; FIG. 4). Further, β-methyl-6-chloromelatonin also blocked the significant increase in PGE₂ levels in 20 ng/ml TNF-α-stimulated EOC-20 cells in a dose-dependent manner (*P<0.01; FIG. 5). Overall, these data indicate that β-methyl-6-chloromelatonin is an effective treatment for neurodegenerative diseases such as ALS, AD, HD and PD.

References

Cacquevel, Mathias. (2004). Cytokines in Neuroinflammation and Alzheimer's Disease. Current Drug Targets, 5(6), 529.

Deckel, A. W. (2001). Nitric oxide and nitric oxide synthase in Huntington's disease. Journal of neuroscience research, 64(2), 99-107.

Hensley, K., et al. (2003). Message and protein-level elevation of tumor necrosis factor alpha (TNF alpha) and TNF alpha-modulating cytokines in spinal cords of the G93A-SOD1 mouse model for amyotrophic lateral sclerosis. Neurobiology of disease, 14(1), 74-80.

McGeer, P., L, & McGeer, G. (2004). Inflammation and neurodegeneration in Parkinson's disease. Parkinsonism & related disorders, 10 Suppl 1, S3-7.

Milstien, S., et al. (1994). Cerebrospinal fluid nitrite/nitrate levels in neurologic diseases. Journal of neurochemistry, 63(3), 1178-1180.

Sargsyan, S., et al. (2005). Microglia as potential contributors to motor neuron injury in amyotrophic lateral sclerosis. Glia, 51(4), 241-253.

Tohgi, H., et al. (1999). Increase in oxidized NO products and reduction in oxidized glutathione in cerebrospinal fluid from patients with sporadic form of amyotrophic lateral sclerosis. Neuroscience letters, 260(3), 204-206.

West, Melinda. (2004). The arachidonic acid 5-lipoxygenase inhibitor nordihydroguaiaretic acid inhibits tumor necrosis factor-A activation of microglia and extends survival of G93A-SOD1 transgenic mice. Journal of Neurochemistry, 91(1), 133.

### Cerebrovascular Disease

The compounds of the present invention are effective in treating cerebrovascular diseases such as subarachnoid hemorrhage, stroke, cerebral infarction intracerebral hemorrhage and cerebral aneurysm. Efficacy was demonstrated employing a well-accepted preclinical model of cerebrovascular disease (Vannucci, 2001). The efficacy of the preferred embodiment of the present invention was demonstrated according to the following protocol.

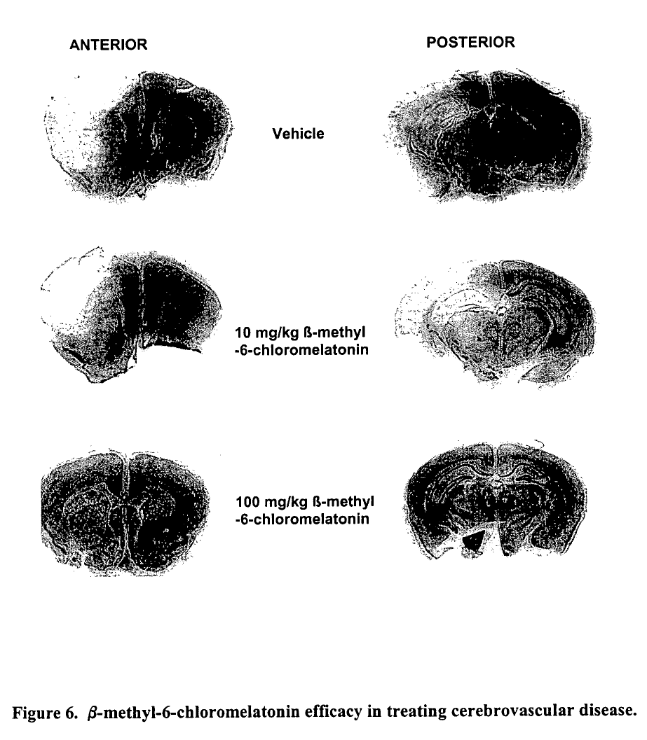

Subjects were 8-12 week old adult male C57B116 mice housed in a temperature- and humidity-controlled vivarium on a 12:12 hr light:dark cycle with food and water available ad libitum. The mice were subjected to ischemia-hypoxia injury (IHI). The injury was rendered by permanently occluding the right common carotid artery and delivering hypoxic gas (7.5% O₂) for 30 minutes employing a gas mask under anesthesia. The animal's body temperature was maintained at 36.5-37.5° C. throughout the experiment. For drug efficacy studies, β-methyl-6-chloro-melatonin was administered intraperitoneally 30 minutes before and 30 minutes after hypoxia at doses of 0 (vehicle only), 10 and 100 mg/kg. On Day 3 after injury, quantitative morphometric image analysis was employed to assess infarction size in Nissl-stained brain sections isolated from the animals. β-methyl-6-chloromelatonin administration caused a 33-40% reduction in brain infarct size in IHI mice compared to vehicle-treated IHI mice (FIG. 6). The protection conferred by β-methyl-6-chloromelatonin was dose-dependent. These data indicate that β-methyl-6-chloro-melatonin blocks IHI-associated tissue damage and is an effective treatment for stroke in this preclinical model.

Reference

Vannucci, S. J., et al., (2001) Experimental stroke in the female diabetic, db/db, mouse. J. Cereb. Blood Flow Metab., 21(1): 52-60.

Treatment of anxiety disorders and affective disorders is a preferred use herein. It is believed that the treatment of traumatic brain injury, Alzheimer's disease and Parkinson's disease is based, at least in part, on the antioxidant and free radical scavenging abilities of the defined compounds.

As discussed above, the defined melatonin derivatives are useful in treating the listed disorders in mammals. Such method comprises administering to a mammal (preferably a human) in need of such treatment a safe and effective amount of one or more of the defined compounds so as to achieve the therapeutic intervention desired. The compounds can be administered by a variety of routes including the oral, rectal, transdermal, subcutaneous, intravenous, intramuscular or intranasal routes. The oral and transdermal routes are preferred. No matter what route of administration is chosen, such administration is accomplished by means of pharmaceutical compositions which are prepared by techniques well known in the pharmaceutical sciences.

As mentioned above, the method of the present invention utilizes pharmaceutical compositions. In making these compositions, one or more of the defined melatonin derivatives (active ingredients) will usually be mixed with a carrier, or diluted by a carrier, or enclosed within a carrier which may be in the form of a capsule, sachet, paper or other container. When the carrier serves as a diluent, it may be a solid, semi-solid or liquid material which acts as a vehicle, excipient or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments, soft and hard gelatin capsules, suppositories, sterile injectable solutions and sterile packaged powders, containing, for example, from about 0.01% to about 10% by weight of the active compound,.

Such carriers are conventional in the pharmaceutical formulation art. Some examples of suitable carriers, excipients, and diluents include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, saline solution, syrup, methylcellulose, methyl- and propylhydroxybenzoates, talc, magnesium stearate and mineral oil. The formulations can additionally include lubricating agents, wetting agents, emulsifying and suspending agents, preserving agents, sweetening agents or flavoring agents. By employing procedures well known in the art, the compositions may be formulated so as to provide rapid, sustained or delayed release of the active ingredient after administration to the patient.

The compositions are formulated, preferably in a unit dosage form, such that each dosage contains from about 0.05 to about 150 mg, more usually from about 20 to about 150 mg, even more usually from about 20 to about 100 mg, of the active ingredient. The term “unit dosage form” refers to physically discrete units suitable as unitary dosages for human subjects and other mammals, each unit containing a predetermined quantity of active material calculated to produce the desired therapeutic effect, in association with one or more suitable pharmaceutical diluents, excipients or carriers.

The compounds employed in the method of the present invention are effective over a dosage range of about 0.1 mg of active ingredient per day to about 150 mg, preferably from about 20 to about 150 mg, even more preferably from about 20 to about 100 mg, of active ingredient per day for treating the listed disorders. Thus, as used herein, the term “safe and effective amount” refers to a dosage range of from about 0.1 to about 150 mg of active ingredient per day. In the treatment of adult humans, the range of about 20 to about 150 mg of active ingredient per day, in single or divided doses, is preferred. However, it will be understood that the amount of compound actually administered will be determined by a physician, in light of the relevant circumstances including the choice of compound to be administered, the chosen route of administration, the age, weight, and response of the individual patient, and the nature and severity of the patient's symptoms.

What is claimed is:

1. A method of treating a patient having a condition selected from anxiety disorders, affective disorders, obesity, intracranial injury, spinal cord injury, neurodegenerative disorders, sclerosis, migraines, fibromyalgia and cerebrovascular disease, comprising the administration to said patient of a safe and effective amount of a melatonin derivative selected from compounds having the formula 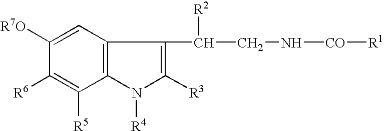
wherein
R¹ is hydrogen, C₁-C₄ alkyl or C₁-C₄ alkoxy;
R² is hydrogen or C₁-C₄ alkyl;
R³ is hydrogen, C₁-C₄ alkyl, phenyl or substituted phenyl;
R⁴ is hydrogen, haloacetyl, C₁-C₅ alkanoyl, benzoyl or benzoyl substituted with halo or methyl;
R⁵ and R⁶ are each individually hydrogen or halo; and
R⁷ is hydrogen or C₁-C₄ alkyl;
provided that when R³, R⁴ and R⁵ are each hydrogen, then R² must be C₁-C₄ alkyl.

2. The method of claim 1 wherein the melatonin derivative is administered at from about 0.1 mg/day to about 150 mg/day.

3. The method of claim 2 wherein the melatonin derivative is selected from compounds of formula (ii).

4. The method of claim 3 wherein R¹ is C₁-C₄ alkyl, R² is hydrogen or C₁-C₄ alkyl, and R⁴ is hydrogen.

5. The method of claim 4 wherein R¹ is methyl.

6. The method of claim 4 wherein R² and R⁷ are independently C₁-C₄ alkyl.

7. The method of claim 6 wherein R² and R⁷ are both methyl.

8. The method of claim 2 wherein the melatonin derivative is selected from N-[2-methyl-2-(5-methoxy-6-fluoroindol-3-yl)ethyl]acetamide; N-[2-ethyl-2-(5-methoxy-6-chloroindol-3-yl)ethyl]acetamide; N-[2-methyl-2-(5-methoxy-6,7-dichloroindol-3-yl)ethyl]acetamide; N-[2-methyl-2-(5-methoxy-6-chloroindol-3-yl)ethyl]acetamide; and mixtures thereof.

9. The method of claim 2 wherein the melatonin derivative is (R)-N-[2-(6-chloro-5 -methoxy-1H-indol-3-yl)propyl]acetamide.

10. The method of claim 9 wherein the melatonin derivative is administered at from about 20 mg/day to about 100 mg/day.

11. The method of claim 2 wherein the melatonin derivative is administered at from about 20 mg/day to about 100 mg/day.

12. The method of claim 1 for treating a patient having a condition selected from anxiety disorders, affective disorders, intracranial injury, spinal cord injury, neurodegenerative diseases, and cerebrovascular disease.

13. The method of claim 12 for treating a patient having a condition selected from anxiety disorders and affective disorders.

14. The method of claim 10 for treating a patient having a condition selected from anxiety disorders and affective disorders.
